(19) United States
(12) Patent Application Publication
(10) Pub. No.: US 2016/0136330 A1
(43) Pub. Date: May 19, 2016

(54) Three-Dimensional Scaffold Functionalized with Micro-Tissues for Tissue Regeneration

(71) Applicants: INSTITUT NATIONAL DE LA SANTE ET DE LA RECHERCHE MEDICALE (INSERM), Paris (FR); UNIVERSITE DE STRASBOURG, Strasbourg (FR)

(72) Inventors: Nadia Benkirane-Jessel, Strasbourg (FR); Sandy Eap, Strasbourg (FR); Laetitia Keller, Strasbourg (FR)

(21) Appl. No.: 14/905,190

(22) PCT Filed: Jul. 16, 2014
(86) PCT No.: PCT/EP2014/065301

§ 371 Date: Jan. 14, 2016

(30) Foreign Application Priority Data
Jul. 17, 2013 (EP) 13306027.7

(51) Int. Cl. A61L 27/38; A61L 27/54; A61L 27/52; A61L 27/18

(52) U.S. Cl. CPC A61L 27/3821; A61L 27/3817; A61L 27/18; A61L 27/54; A61L 27/52; A61L 2300/64; A61L 2300/412; A61L 2430/02; A61L 2430/06

(57) ABSTRACT

The present invention concerns a biomaterial devoid of a growth factor, comprising: —a three-dimensional scaffold made of a biocompatible polymer; and—living cells, wherein said living cells are in form of microtissues and the nanofibrous three-dimensional scaffold is a nanofibrous scaffold. It further concerns a method for manufacturing such a biomaterial. Finally, it concerns such a biomaterial for use in the treatment of a bone and/or cartilage defect.

### TECHNICAL FIELD OF THE INVENTION

The present invention concerns biomaterials comprising a three-dimensional functionalized scaffold and living cells useful for tissue generation. It further concerns a method for producing such biomaterials.

It also concerns methods of treating bone or cartilage defects using such biomaterials and biomaterials for use in the treatment of bone or cartilage defects, in particular as implants.

### BACKGROUND OF THE INVENTION

Bone regeneration is a complex, well-orchestrated physiological process of bone formation, which occurs during normal fracture healing, and is involved in continuous remodelling throughout adult life. In the clinic, bone regeneration may be required in large quantity, such as for skeletal reconstruction of large bone defects created by trauma, tumour resection, or cases in which the regenerative process is compromised (non-unions, osteoporosis). Current bone-regeneration processes, including the free fibula vascularised graft, autologous bone graft, allograft implantation, and use of growth factors, scaffolds and osteo-progenitor cells are unsatisfactory as they yield insufficient quantities of bone.

A promising strategy for bone regeneration is the tissue-engineering approach, which aims to generate new functional tissues, rather than just to implant non-living scaffolds. In bone-tissue engineering, progenitor cells, such as mesenchymal stem cells (MSCs; native or expanded) or mature cells (osteoblasts) seeded in biocompatible scaffolds and ideally in three-dimensional tissue-like structures with appropriate growth factors such as bone morphogenic proteins (BMPs) in order to generate and maintain bone. Current approaches do not allow sufficiently robust bone regeneration. Further, the speed of bone regeneration needs to be improved.

It has been previously shown that adding osteoblasts cells may be useful in enhancing the speed of bone regeneration. The patent application WO2012/113812 A1 discloses a biomaterial comprising a nanofibrous scaffold of biodegradable polymers coated with polyelectrolyte multilayers which may incorporate a growth factor. When living cells are deposited on such nanostructured hybrid membranes, the smart nano-reservoirs of growth factors formed by the polyelectrolyte multilayers allow for slow and controlled release over the period of tissue re-growth.

Recently, it was shown that such a biomaterial with a thickness of 50 μm allows excellent bone regeneration with complete colonisation and bone induction inside the scaffold after in vivo implantation in mice (Mendoza-Palomares, C. et al., ACS Nano 6, 483-490 (2012)).

This technology is very promising for bone regeneration for small lesions. However, it appears to be limited to implants having a thickness of up to 50 μm, as cell colonisation and bone induction are not as satisfactory for thicker implants.

Lai et al. (2011 PLoS ONE 6(10): e26821. doi:10.1371/journal.pone.0026821) describes microtissue formation upon culturing neural cells on three-dimensional polystyrene scaffolds.

There thus still remains a need for biomaterials allowing efficient bone and/or cartilage repair including for large lesions, such as for the restoration of the entire osteochondral unit. Such biomaterials should enable high quality, robust, durable and rapid tissue regeneration to shorten recovery times and decrease risks of postoperative complications for patients. Ideally, these biomaterials should allow for fast and in depth tissue regeneration without the need for a growth factor. Also, it would be advantageous to shorter production times so as to lower costs and to compete with conventional interventions.

### SUMMARY OF THE INVENTION

According to the invention, this need is met by a biomaterial comprising a nanofibrous scaffold which is functionalized by microtissues.

Such biomaterials advantageously create a second three dimensional tissue regeneration system within the three dimensional scaffold.

It was discovered that such scaffolds allow for accelerated regeneration of living cells deposited thereon. Also, the tissue regeneration was shown to proceed in depth, up to the core of a scaffold. Tissue generation upon such biomaterials thus does not require the presence of a growth factor.

It has further been demonstrated that such functionalized nanofibrous scaffolds are capable of efficiently inducing bone regeneration in vivo.

The biomaterials according to the present invention represent a new generation of biomaterials for regenerative nanomedicine. Indeed, the strategy of functionalizing the scaffold by microtissues affects cell proliferation and thereby allows for complete colonisation even of thick scaffolds, which enhances the efficacy compared to scaffolds functionalized with living cells used in the clinic today. It is presently thought that this improvement is due to the replacement of the bidimensional cell monolayer formed by living cells on the scaffold by a further three-dimensional environment for the cells formed by the microtissues.

According to a first aspect, the invention thus provides a biomaterial devoid of a growth factor, comprising:
a three-dimensional scaffold made of a biocompatible polymer; and
living cells,
   wherein said living cells are in form of microtissues.

Preferably, the three-dimensional scaffold is a nanofibrous scaffold or a hydrogel.

The biocompatible polymer is preferably selected from poly(ε-caprolactone), poly-(lactic acid), poly(glycolic acid), poly(ethylene glycol) terephthalate, poly(butylene terephthalate), collagen, fibrin, hyaluronic acid, hydroxyapatite, chondroitine sulfate, chitosan, copolymers and mixtures thereof.

The three-dimensional scaffold has a preferred thickness of 50 μm to 2 cm.

The microtissues may comprise in particular osteoblasts, endothelial cells, keratinocytes, myocytes, embryonic stem cells, mesenchymal stem cells and/or chondrocytes.

The microtissues may have in particular a size of 100 to 300 μm.

Particularly preferred is such a biomaterial which is an implant.

According to a second aspect, the invention provides a method for producing a biomaterial comprising a three-dimensional scaffold made of biocompatible polymer and living cells, comprising the steps of:
(a) producing a three-dimensional scaffold made of biocompatible polymer; and
(b) contacting said three-dimensional scaffold with microtissues of living cells so as to form a functionalized three-dimensional scaffold.

The biocompatible polymer may be selected in particular from poly(ε-caprolactone), collagen, fibrin, poly-(lactic acid), poly(glycolic acid), poly(ethylene glycol) terephthalate, poly(butylene terephthalate) or co-polymers thereof.

The microtissues may comprise in particular osteoblasts, endothelial cells, keratinocytes, myocytes, embryonic stem cells, mesenchymal stem cells and/or chondrocytes.

Step (b) of the method above is preferably carried out by injection or deposition of a solution or dispersion of microtissues into or onto the three-dimensional scaffold or by dipping the three-dimensional scaffold into said solution or dispersion.

According to a third aspect, the invention concerns such a biomaterial for use in the treatment of a bone and/or cartilage defect, in particular for use in the treatment of a bone and/or cartilage defect in a patient suffering from osteochondritis dissecans, osteonecrosis, osteochondral fracture(s), spinal fusion, a bone and/or cartilage defect due to an injury, a bone and/or cartilage defect due to ageing, a bone and/or cartilage defect necessitating maxillofacial reconstruction, a bone and/or cartilage defect necessitating sinus lift, a bone and/or cartilage defect necessitating alveolar ridge augmentation, or bone and/or cartilage loss due to a tumor.

Also concerned is such a biomaterial for use in the treatment of a subchondral bone defect or of an osteochondral defect.

Particularly preferred is such a biomaterial for use as an implant.

### DEFINITIONS

Within the present application is meant by “biomaterial” any material suitable for use in vivo in mammals, in particular in human patients. More specifically, the biomaterials according to the invention are implantable and thus suitable for use as implants.

The term “three-dimensional scaffold” means a matrix that is capable of mimicking the natural properties of a tissue (e.g. of bone and/or cartilage) while providing a temporary scaffold for tissue regeneration. This term does not encompass endogenous extra-cellular matrices.

The term “nanofibrous scaffold” refers to a specific three-dimensional scaffold formed by fibers, notably polymeric fibers, whose diameter is less than 1 μm. Due to the presence of fibers, nanofibrous scaffolds not only mimic the three-dimensional structure of a tissue, but also facilitate adhesion and spreading of cells.

The term “hydrogel” refers to a solid, jelly-like material which exhibits no flow when in the steady-state. Such hydrogels are made of a network of cross-linked hydrophilic polymer chains in water as the dispersion medium. In hydrogels, the polymer chains form the three-dimensional structure which guides the adhesion and spreading of cells.

The term “living cells” refers to cells which are capable of growth, proliferation and differentiation.

The term “microtissues” designates multicellular spheroids of living cells which are morphologically and functionally very similar to native tissue. Microtissues show close cell-cell contacts, a gene expression profile close to in vivo, an intact endogenous extracellular matrix and physiological nutrient and oxygen gradients.

As used throughout the present specification, the term “therapeutic molecule” refers to any molecule intended to treat or prevent a disease. It may for example correspond to a drug for which a marketing approval has been issued (e.g. by the European Medicines Agency (EMA) or by the U.S. Food and Drug Administration (FDA)), or to a candidate drug undergoing clinical or pre-clinical trials. The therapeutic molecule may for example correspond to a polypeptide (including recombinant proteins, antibodies and peptides), a nucleic acid (including RNA and DNA molecules), a chemical molecule (e.g. a small molecule), or a sugar (e.g. a lipopolysaccharide).

The term “biomaterial devoid of a growth factor” refers to a biomaterial devoid of added growth factor.

### DETAILED DESCRIPTION OF THE INVENTION

The subject-matter of the present invention is a biomaterial devoid of a growth factor, comprising:
a three-dimensional scaffold made of a biocompatible polymer; and
living cells,
   wherein said living cells are in form of microtissues.

Indeed, such a biomaterial is particularly useful as a new strategy to regenerate robust tissue including for thick scaffolds such as required for the repair of large lesions.

[Biomaterials According to the Invention]

The biomaterial according to the invention comprises a three-dimensional scaffold made of a biocompatible polymer.

Said a biocompatible polymer may either be synthetic or natural. The biocompatible polymers are preferably biodegradable. However, three-dimensional scaffolds made of non-biodegradable biocompatible polymers are also contemplated herein since such scaffolds are useful e.g. when carrying out a spinal fusion, in replacement of a prosthesis, or as a bone defect filling material.

Said three-dimensional scaffold is preferably porous, or comprises at least one porous side or part.

The three-dimensional scaffold may be notably formed by large molecules such as polymers or by thin fibers such as nanofibers. Hence, the scaffold may be in particular a gel, notably a hydrogel or a nanofibrous material.

Hydrogels are well-known to the skilled in the art. A collagen hydrogel may for example be prepared by mixing collagen (e.g. 3 mL of Rat Tail Type-I Collagen) with a medium containing 10% FBS (e.g. 5.5. mL) and with a 0.1 M NaOH solution (e.g. 0.5. mL). An alginate hydrogel may for example be a mixture of alginate and hyaluronic acid (e.g. a alginate:hyaluronic acid solution (4:1), which may be prepared in a 0.15 M NaCl solution at pH 7.4).

Nanofibrous scaffolds are based on fibers typically having a diameter of about 50 to about 1000 μm, preferably of about 50 to about 500 μm or of about 100 to about 1000 μm, and which form a material with a high porosity and an interconnected pore structure. Such material is particularly suitable as a scaffold structure notably because of its high specific surface area.

Nanofibrous scaffolds suitable for use as implants are well-known to the skilled in the art. For example, Swieszkowski et al. (2007 Biomol Eng. 24:489-95) discloses several nanofibrous scaffolds suitable for use as implants in the frame of bone and/or cartilage regeneration.

These nanofibrous scaffolds made of biodegradable polymers can for instance be made of poly(ε-caprolactone), poly(lactic acid), poly(glycolic acid), poly(ethylene glycol)-terephtalate, poly(butylenes terephtalate), or co-polymers or mixtures thereof.

The nanofibrous scaffold can also be made of polymers such as collagen, fibrin, hyaluronic acid, hydroxyapatite, chondroitine sulfate, chitosan, and mixtures thereof.

In a preferred embodiment, the nanofibrous scaffold comprises or consists of poly(ε-caprolactone) (PCL).

In the frame of the present invention, polymer nanofibers are obtained preferably by electrospinning. Particularly preferred are nanofibrous scaffolds made from electrospun PCL (Poly (ε-caprolactone) nanofibers as obtained as described in Li et al. (2005 Biomaterials. 26:599-609) or in Savarino et al. (2007 Biomaterials. 28:3101-9).

Electrospun nanofibers have extremely high specific surface area thanks to their small diameters and allow the scaffold to mimic the collagen extracellular matrix. Such nanofibrous materials are highly porous (above 90%) with excellent pore interconnectivity and superposition of thin nanofibrous layers. Interactions between the different nanofibrous layers occurred randomly during the electrospinning process which leads to a random distribution of the pores in term of size and because of punctual interactions between different nanofibrous layers. These unique characteristics reveal nanofibrous biomaterials with many desirable properties for advanced applications.

In another preferred embodiment, the scaffold comprises or consists of collagen. Collagen is a natural polymer that can for example be obtained from pig. Nanofibrous scaffolds made of collagen are commonly used as implants, and include e.g. the Bio-Gide® resorbable collagen membrane commercialized by Geistlich Pharma AG (Germany).

Within the present invention, the three-dimensional scaffold mimics the three-dimensional structure of bone and/or cartilage.

The biomaterials functionalized with microtissues according to the invention are particularly interesting because they allow for easy and fast in-depth cell colonisation of the scaffold. Such colonisation allows the spontaneous migration of cells throughout the nanofibrouse three-dimensional scaffold without use of external forces such as a pulsatile flow or an injection under pressure. Therefore, the invention is particularly advantageous for the preparation of thick biomaterials, i.e. when the scaffold has a thickness of above 50 μm, advantageously up to 50 mm, and most preferred from 0.1 to 20, and in particular from 0.5 to 10 mm.

In the frame of the present invention, the three-dimensional scaffold is functionalized with living cell microtissues. Indeed, implanting living cells is a promising solution to tissue or organ repair. Many types of cells can aggregate and differentiate into three-dimensional multi-cellular spheroids when cultured in suspension or a non-adhesive environment. Compared to conventional monolayer cultures, microtissues resemble real tissues better in terms of structural and functional properties.

In view of bone and/or cartilage regeneration, said living cell microtissues may for example comprise or consist of osteoblasts, chondrocytes, stem cells (e.g. embryonic or mesenchymal stem cells), bone marrow stromal cells, or a mixture thereof. Preferably, said living cell microtissues comprise or consist of osteoblasts, chondrocytes, or a mixture thereof. In a specific embodiment, embryonic stem cells may be excluded from the living cell microtissues according to the invention.

The preparation of microtissues is known in the art, and described in detail notably in WO 2012/014047 A1. Particularly preferred in the framework of the present invention is the preparation of microtissues in an automated hangingdrop platform, using a GravityPLUS™ plate (sold by InSphero, Zurich, Switzerland). Such microtissues are well calibrated in size, form and number of cells and thus ensure a homogeneous colonisation and subsequent tissue growth. Microtissues are also available commercially, notably at InSphero, Zurich, Switzerland.

Said living cell microtissues are preferably made of human cells, and most preferably autologous cells (i.e. cells that are obtained from the patient to be treated).

In a specific embodiment, said living cell microtissues are suspended in a hydrogel (e.g. an alginate hydrogel or a collagen hydrogel) that is contacted with the three-dimensional scaffold. In other terms, the biomaterial according to the invention may also comprise in addition to the scaffold, a hydrogel comprising living cell microtissues.

In a preferred embodiment of the invention, the biomaterial comprises or consists of:
a three-dimensional scaffold in form of a nanofibrous scaffold made of electrospun PCL (Poly (ε-caprolactone) nanofibers; and
microtissues of osteoblasts, chondrocytes, or a mixture thereof.

According to an embodiment of the present invention, the three-dimensional scaffold may further also comprise a therapeutic molecule other than a growth factor and thus also serve as a reservoir for the therapeutic molecule. Such a scaffold further functionalized with a therapeutic molecule, allows for sustained release of said therapeutic molecule at the site of implantation of the biomaterial according to the invention.

[Methods for Producing Biomaterials According to the Invention]

According to another aspect, the invention pertains to a method for producing the biomaterial as described above, said method comprising the steps of:
producing a three-dimensional scaffold made of biocompatible polymer; and
contacting said three-dimensional scaffold with microtissues of living cells so as to form a functionalized three-dimensional scaffold.

With regard to the first step of the method above, several methods for producing three-dimensional scaffolds are known in the art. With regard to nanofibrous scaffolds, it may be referred to Swieszkowski et al. (2007 Biomol Eng. 24:489-95). Hydrogels and their methods of preparation are also known as such.

The second step of the method above, the contacting of the scaffold with microtissues so as to form a functionalized scaffold, may be carried out in different ways.

Generally, the microtissues are used in form of a suspension in a liquid medium, generally an aqueous solution and most often a buffer solution. Alternatively, as explained above, the microtissues may also be introduced into a hydrogel before contacting with the scaffold.

A very convenient and simple means to put the scaffold in contact with the microtissues is to deposit the suspension of microtissues onto the scaffold, for instance by spraying, or to dip the scaffold into the suspension and to let the suspension impregnate the scaffold with the assistance of capillary forces.

Another option is to force the suspension of microtissues into the scaffold by way of injection, using a syringe for instance.

Before use, the biomaterial according to the invention may be equilibrated (e.g. by bringing it in contact with serum-free medium).

Advantageously, the biomaterial according to the invention may be produced without adding growth factor.

The invention further provides biomaterials obtainable by the methods described herein.

[Therapeutic Uses of the Biomaterials According to the Invention]

The inventors have shown that the biomaterials according to the invention, functionalized with microtissues, are very efficient in inducing bone and/or cartilage regeneration. In particular, they are suitable for use as implants.

Therefore, the invention pertains to the biomaterial described in the above paragraphs, for use as a bone and/or cartilage defect filling material, or for use in bone and/or cartilage regeneration. The invention also provides the biomaterial described in the above paragraphs, for use in the treatment of a bone and/or cartilage defect.

The bone and/or cartilage defect may affect either the bone, or the cartilage, or both. It may for example be a chondral defect, an osteochondral defect, or a subchondral bone defect.

In a specific embodiment according to the invention, the bone and/or cartilage defect is a subchondral bone defect. The invention thus provides a biomaterial described in the above paragraphs for use in subchondral bone regeneration and/or for use in the treatment of a subchondral bone defect.

In particular, the biomaterial according to the invention finds use in the treatment of bone and/or cartilage defect(s) in patients suffering from osteochondritis dissecans, osteonecrosis, osteochondral fracture(s), spinal fusion, a bone and/or cartilage defect due to an injury (e.g. a sport injury or an injury due to an accident), a bone and/or cartilage defect due to ageing, a bone and/or cartilage defect necessitating maxillofacial reconstruction, a bone and/or cartilage defect necessitating sinus lift, a bone and/or cartilage defect necessitating alveolar ridge augmentation, or bone and/or cartilage loss due to a tumor (including benign and cancerous tumors).

In a specific embodiment, the bone and/or cartilage defect is an articular defect, such as e.g. a defect of the knee and/or of the ankle.

Due to the presence of living microtissues, cell colonisation proceeds in depth even for thick scaffolds. The biomaterial of the invention is thus particularly appropriate for the treatment of large and/or deep bone and/or cartilage defect.

For instance, when the biomaterial is for use as an implant in the treatment of a large and/or deep bone defect, the biomaterial preferably comprises osteoblasts. When the biomaterial is for use as an implant in the treatment of a large and/or deep cartilage defect, the biomaterial preferably comprises chondrocytes. When the biomaterial is for use as an implant in the treatment of large and/or deep defects affecting the bone and the cartilage (e.g. an osteochondral defect or a subchondral bone defect), the biomaterial preferably comprises both osteoblasts and chondrocytes.

The invention thus provides a method for treating a bone and/or cartilage defect, comprising the step of implanting the biomaterial according to the invention in an individual in need thereof.

The individual and/or patient to be treated is preferably a human individual and/or patient. However, the biomaterials according to the invention also find use in the field of veterinary medicine.

All references cited herein, including journal articles or abstracts, published patent applications, issued patents or any other references, are entirely incorporated by reference herein, including all data, tables, figures and text presented in the cited references.

Although having distinct meanings, the terms “comprising”, “having”, “containing’ and “consisting of” may be replaced with one another throughout the above description of the invention.

In the frame of the present description, all molecules and cells may optionally be isolated and/or purified.

The invention will be further evaluated in view of the following examples and figures.

### BRIEF DESCRIPTION OF THE FIGURES

FIG. 1: Light micrographs of PCL (poly(ε-caprolactone)electrospun nanofibrous scaffold according to Example 1: front view (A), three-quarter view (B), front view from a transmitted light (C).

FIG. 2: Fluorescence micrographs of human primary osteoblasts microtissue stained with DAPI (DNA) and phalloidin (actin) (A, B, C) and of the living microtissue nesting in the scaffold according to Example 2 (D).

FIG. 3: SEM visualization and morphology of human osteoblasts microtissues growing onto the scaffold according to Example 2 after 7 days (A), and 14 days (B and C).

FIG. 4: In vitro proliferation of human osteoblasts single cells and microtissues growing on the surface of the scaffold according to Example 1 after 3 days, 7 days, 14 days, 21 days and 28 days (A). Bone induction and mineralization (alizarin red staining) after 21 days in vitro culture of human osteoblasts single cells (C) and microtissues after 21 days (D) and 28 days (E). Control without cells (B).

FIG. 5: In vivo bone induction of the scaffold functionalized with human primary osteoblasts single cells (according to Comparative Example, A and C) and microtissues (according to Example 2, B and D) after 4 weeks calvaria implantations in nude mice. For Mallory staining (A and B): cell nucleus in dark grey, bone collagen matrix in light grey, HB: host bone, NB:new bone. For calcein staining (C and D): cell nucleus in dark grey with DAPI, bone in formation in light grey with calcein.

FIG. 6: Schematic representation of an osteochondral implant prepared according to example 4.

### EXAMPLES

### Example 1

### Material and Methods

Chemicals.

Poly(ε-caprolactone) (PCL), Capa 6800 (Mw=80000) analytical grade, was obtained from Perstorp (Industriparken, Sweden). PCL was dissolved in a mixture of dichloromethane/dimethylformamide (DCM/DMF 40/60 vol/vol) at 27% wt/vol and was stirred overnight before use to ensure good polymer solubilisation.

Electrospinning

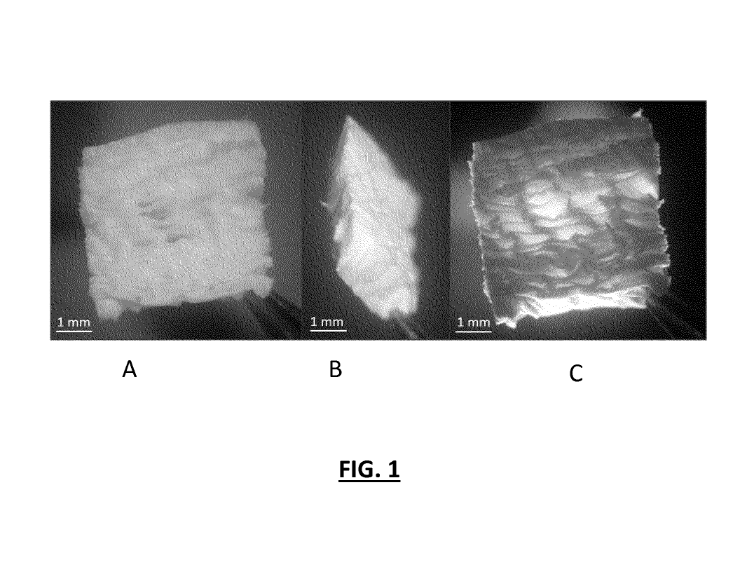

A standard electrospinning set-up Apparatus EC-DIG purchased from IME Technologies (Eindhoven, Netherlands) was used to fabricate the PCL three-dimensional nanofibrous scaffolds. The PCL solution was poured into a 5 mL syringe and ejected through a 21G needle of 0.8 mm diameter at a flow rate of 1.2 mUh thanks to a programmable pump (ProSense). The electrospun hjet was focused thjanks to the use of a poly(methyl metacrylate) (PMMA) plate of 2.5 mm thick pierced with a hole (25 mm in diameter) placed over the conductive collector. The collector was placed at a distance from the needle of 16 cm. A voltage of +15 kV was applied on the needle whereas −5 kV was applied on the collector during the electrospinning process. For all the experiments, the temperature and humidity were kept constant at 25° C. and 40%. The three-dimensional scaffold obtained by electrospinning is shown on FIG. 1.

Cells Culture.

Human primary osteoblasts were obtained from PromoCell (Heidelberg, Germany) and cultured in a specific osteoblast growth medium added with complement and 50 U mL⁻¹ penicillin, 50 μg mL⁻¹ streptomycin, 2.5 μg mL⁻¹ amphotericin B. The cells were incubated at 37° C. in a humidified atmosphere of 5% CO₂. When cells reached sub-confluence, they were harvested with trypsin and sub-cultured.

Microtissues Culture.

Osteoblasts were seeded in GravityPLUS™ plate (from InSphero, Zurich, Switzerland) to produce microtissues. 1×10⁴ cells per microtissue were seeded in these plates and cultivated during 5 days. For bone regeneration, osteoblasts microtissues were then seeded onto the PCL three-dimensional scaffolds for in vitro studies and in vivo implantations.

Alginate Hydrogel Culture

Chondrocytes were seeded in GravityPLUS™ plate (from InSphero, Zurich, Switzerland) to produce microtissues. 1×10⁴ cells per microtissue were seeded in these plates and cultivated during 5 days. For cartilage regeneration, chondrocytes microtissues were then suspended in a solution of alginate (12 mg mL⁻¹)/hyaluronic acid (3 mg mL⁻¹) hydrogel. The alginate was then polymerized in a solution of CaCl₂ at 102 mM during 15 minutes at 37° C. The microtissues embedded with alginate/AH were then cultured in medium complemented with 1 mM of CaCl₂.

Cell Viability and Proliferation.

Cell viability was determined by trypan blue exclusion. AlamarBlue® (Serotec) was used to assess cellular proliferation. The Alamar Blue test is a non-toxic, water-soluble, colorimetric redox indicator that changes color in response to cell metabolism. In this study, 4×10⁴ human osteoblasts single cells were seeded on top of PCL scaffolds (n=3) placed on 48-well plates, and 4 microtissues of 1×10⁴ osteoblasts were seeded onto other PCL scaffolds (n=3) for comparison. After 3 days of culture, cells were incubated in 10% AlamarBlue/DMEM solution in a humidified atmosphere at 37° C. and 5% CO₂. After 4 hours, 200 μL of incubation media was transferred to 96-well plates and measured at 570 nm and 630 nm in order to determine the percentage of Alamar Blue reduction. The same protocol has been then reiterated after 7, 14, 21 and 28 days of culture to monitor cell proliferation.

In Vitro Analysis of Mineralization Using Alizarin Red S Staining.

Alizarin Red S powder was dissolved in distilled water in a concentration of 2 g for 100 mL. The samples were incubated in the Alizarin Red solution for 20 min and then rinsed with distilled water several times. The samples were embedded in Tissue-Tek OCT™ Compound to be cut in sections (35 μm) with a cryostat (LEICA JUNG CM 3000). The sections were then observed under the optical microscope (LEICA DM 4000 B).

In Vivo Implantation in Calvaria of Nude Mice.

The mice were anesthetized and implanted with a PCL three-dimensional scaffold seeded with human single cell osteoblasts on one side of calvaria and a PCL three-dimensional scaffold seeded with osteoblasts microtissues on the other side. Five mice per group were used. Implantations have been carried out with the same protocol as above. After 2 or 4 weeks of calvaria implantation, the mice were sacrificed and the samples extracted for analysis.

In Vivo Subcutaneous Implantation in Nude Mice.

The mice were anesthetized and implanted with a PCL three-dimensional scaffold seeded with human single cell osteoblasts and a PCL three-dimensional scaffold seeded with osteoblasts microtissues. The samples were implanted between skin and muscles behind the ears of mice. Five mice per group were used. After 4 weeks of calvaria implantation, the mice were sacrificed and the samples extracted for analysis.

Histological Analysis of Bone Induction Mallory Coloration.

The implants were fixed with Bouin Hollande solution during two days. Then, they were dehydrated through a series of increasing ethanol concentrations, cleared with toluene and embedded in paraffin wax. Sections were cut at 7 μm using a sledge microtome and mounted on glass slides. After the removal of paraffin wax, sections of the calvaria and subcutaneous implants were stained using Mallory coloration during two days.

Scanning Electron Microscopy (SEM).

To analyze morphology, after fixation in 2.5% glutaraldehyde for 2 h at 37° C., osmium tetroxide 1% incubation for 1 h and dehydration, the scaffolds were gold-coated (Edwards Sputter Coater) and observed with a scanning electron microscope (SEM Hitachi TM1000) in conventional mode (high vacuum).

Confocal Microscopy.

Human osteoblast single cells or microtissues were seeded onto PCL three-dimensional electrospun scaffolds and cultivated for 1 and 21 days before fixation with PFA 4%. Then, cell nuclei were stained with DAPI and nanofibers were stained with PLL-FITC. Fluorescence observations were performed with a confocal microscope Zeiss LSM 700.

Immunofluorescence staining and observation.

Samples Seeded with Human Osteoblast as Single Cells or Osteoblasts Microtissues were cultivated for 21 days. Then, they were fixed with 4% PFA over 1 h, permeabilized with 0.1% Triton X-100 for 1 h and incubated for 30 min with Alexa Fluor 546-conjugated phalloidin (Molecular Probes) for F-actin labelling and 5 min with 200 nM DAPI (Sigma) for nuclear staining. Bone growth induction was measured by assaying expression of osteocalcin and BSPII, using polyclonal goat anti-oscteocalcin ( 1/200; Santa Cruz Biotechnology) and monoclonal mouse anti-BSPII ( 1/200; Santa Cruz Biotechnology) overnight at 4° C. After washing with PBS, the samples were incubated with secondary anti-goat or anti-mouse antibodies conjugated to Alexa Fluor 488 (Invitrogen). The samples were observed under a fluorescent microscope (LEICA DM 4000 B).

Calcein Injection and Observation.

Subcutaneous injections of calcein (10 mg/kg, Sigma) in PBS were given on the tenth and third days before necropsy. After 4 weeks of calvaria implantation, the mice were sacrificed and the samples extracted. The samples were then embedded in Tissue-Tek, frozen at −20° C. and cut in sections with a cryostat (Leica JUNG CM3000). Cell nuclei were stained with 200 nM DAPI (Sigma) for 5 min. The sections were mounted with anti-fading medium and observed under a fluorescent microscope (LEICA DM 4000 B).

### Comparative Example

### Scaffold Functionalized with Living Osteoblasts

In this study, the three-dimensional scaffold was an electrospun PCL (Poly (ε-caprolactone) nanofibers implant (689±45 μm fiber diameter) with a thickness of 1 mm was manufactured (FIG. 1). Human primary osteoblasts after Live-Dead fluorescent staining were visualized before incorporation into the three-dimensional implants.

The capability of this electrospun nanofiber scaffold to induce bone regeneration when seeded by human osteoblasts was analyzed in vitro. The cell morphology was followed by SEM and showed an increase of cell adhesion with time.

The results show that the infiltration of the cells into the porous structure was relatively limited, and resulted in the formation of a monolayered cell film onto the upper surface of the scaffold, but did not lead to any notable cell infiltration into the pores.

As mentioned above, excellent bone regeneration with complete colonisation and bone induction inside the scaffold after implantation (mouse calvaria, 4 weeks) was observed recently for such an implant with a thickness of 50 μm (Mendoza-Palomares, C., et al., ACS Nano 6, 483-490 (2012)).

However, such a scaffold with a thickness of around 1 mm as prepared in this example, bone induction was still limited to the outer surface even after 4 weeks implantation.

### Example 2

### Scaffold Functionalized with Microtissues (In Vitro)

The comparative Example was repeated, except that the scaffold was seeded with living human osteoblasts microtissues instead of living human osteoblasts single cells.

The microtissues were prepared according to the protocol indicated above, using a GravityPLUS™ plate for hanging-drop cell culture from InSphero AG (Zurich, Switzerland).

The incorporation, cell colonization, proliferation and bone induction by the living human osteoblasts microtissues into the scaffold was studied.

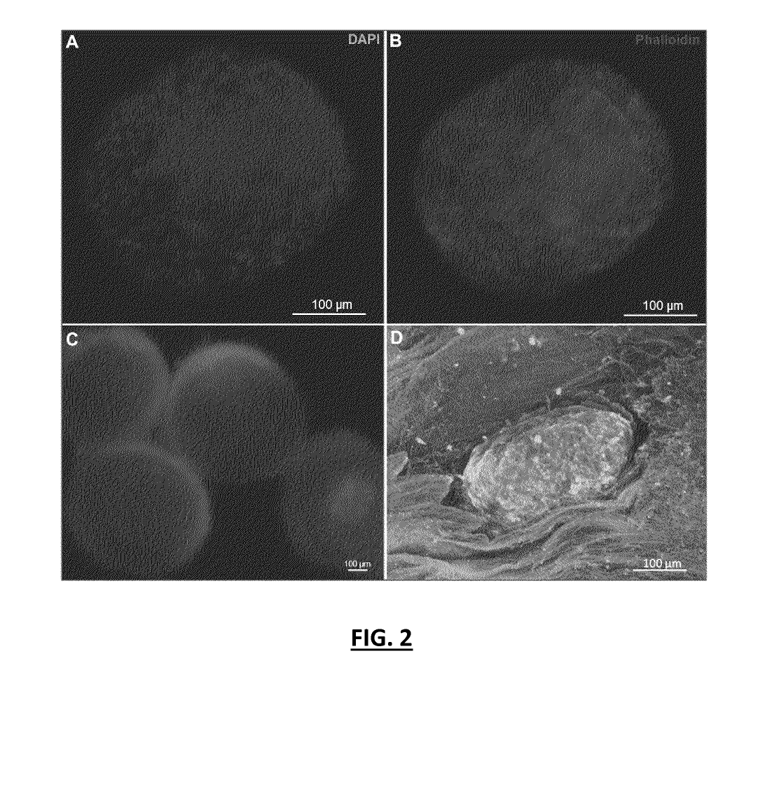

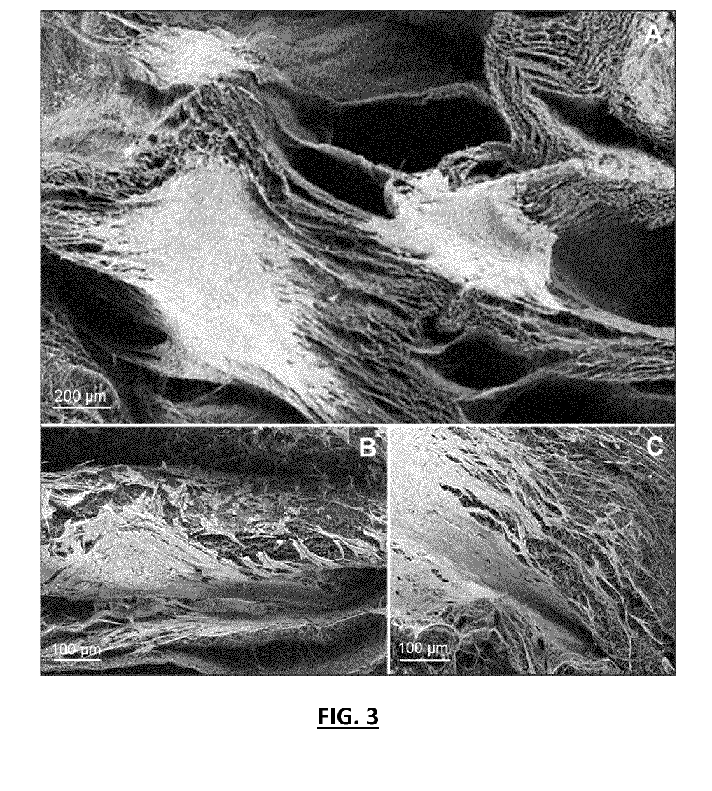

In particular, the behavior of the cells in contact with the scaffold observed by fluorescence microscopy showed that the microtissues remain viable after incorporation into the scaffold (FIG. 2D). The behavior of the osteoblasts microtissues and the capability of the functionalized scaffold to induce bone regeneration after colonization and proliferation were studied by SEM. The behavior of the microtissues with time after the deep colonization of the scaffold was observed by scanning electronic microscopy (SEM) (FIG. 3).

The results indicate that the microtissues seeded onto the scaffold spread into the scaffold with time. After 7 days, the microtissues adhere onto the scaffold and have even begun to spread, showing the osteoblast migration onto the nanofibers (FIG. 3A). Between day 7 and day 14, the osteoblasts pursue colonization of the scaffold and infiltration into the pores. Surprisingly, a layer by layer colonization is observed, presumably due to the guiding influence of the nanofibers on the osteoblasts: cells follow the fiber organization in the scaffold using their elongated filopodia (FIGS. 3B and 3C). When using the single cells, this phenomenon was not observed.

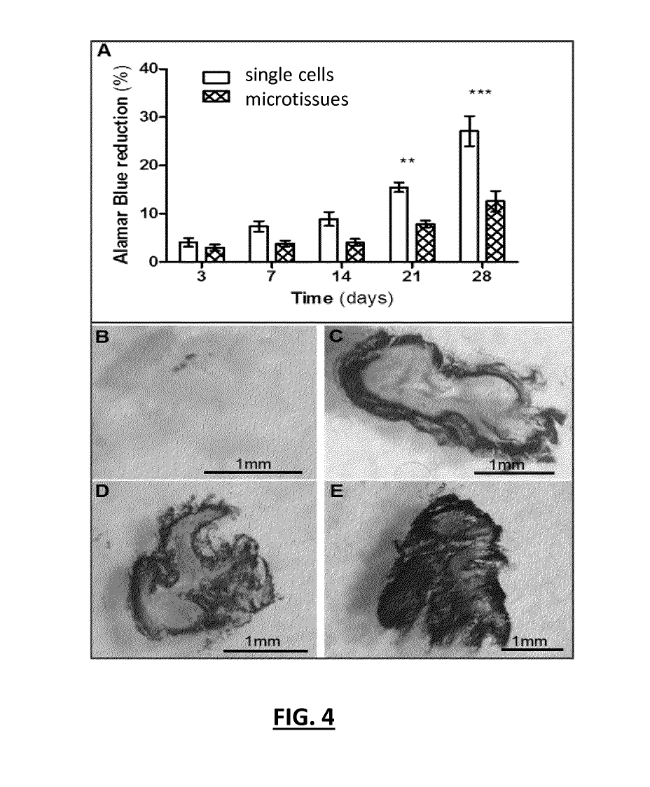

The proliferation and cell activity of human osteoblasts microtissues seeded on the electrospun nanofiber thick scaffold as also followed during 28 days and compared to a scaffold functionalized with a human osteoblast single cells (FIG. 4).

The results show that in scaffolds functionalized with osteoblast single cells, cell proliferation increases with time (day 3 until day 28) (FIG. 4A). Using osteoblasts microtissues, cell proliferation also increases, albeit somewhat slower. This phenomenon is presumably due to the fact that colonization into the scaffold functionalized with microtissues occurs before proliferation (FIG. 4A). These results are in line with bone induction and mineralization observed by alizarin red staining after 21 days in vitro culture of scaffolds functionalized with a human osteoblast single cells (FIG. 4C) and microtissues (FIG. 4D). After 28 days, the mineralization of scaffolds functionalized with osteoblast single cells is still limited to the border, as shown in FIG. 4C, while full mineralization is already achieved in scaffolds with microtissues (FIG. 4E).

### Example 3

### Scaffold Functionalized with Microtissues (In Vivo)

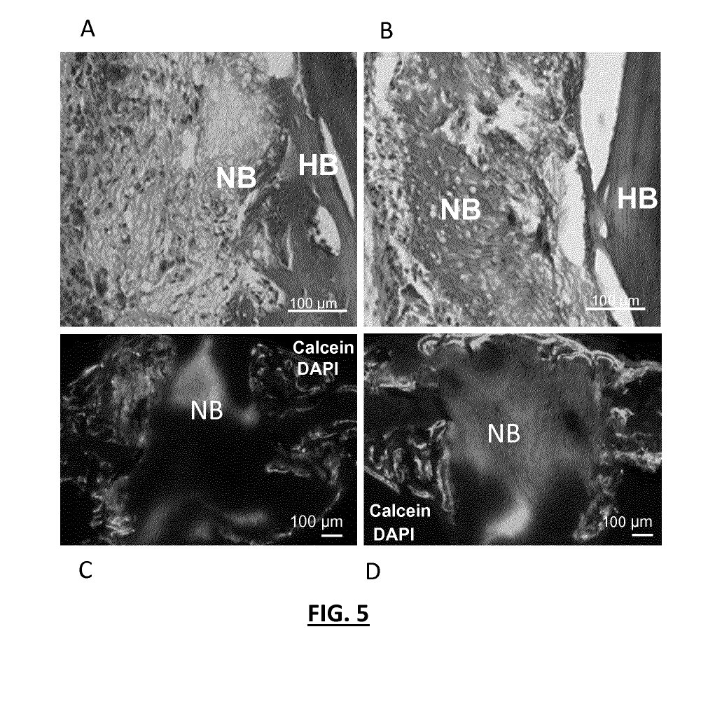

Using Thick scaffolds functionalized with osteoblasts microtissues prepared in Comparative Example and in Example 2, subcutaneous and calvaria implantations were realized in nude mice (FIG. 5A-D).

The results for the scaffolds functionalized with the living microtissues showed that, after 4 weeks, cells have migrated into the scaffold, colonizing the scaffold even deep within, and also show evidence that bone induction has occurred (FIGS. 5B and 5D).

It is further noted that bone induction is faster for scaffolds functionalized with microtissues in comparison to scaffolds functionalized with osteoblast single cells (FIGS. 5C and 5D). Indeed, the comparison of the amount of mineralized matrix yields 8% mineralized area for the scaffold functionalized with osteoblast single cells against 22% mineralized area for the scaffold functionalized with microtissues.

Further, mice calvaria implantations have been carried out with the same conditions in nude mice to confirm the subcutaneous results.

After 2 weeks, both implants showed good osteo-integration, large cell colonization and even mineralized bone matrix. The scaffold functionalized with microtissues presented a large newly mineralized area.

These results indicate that the biomaterial according to the invention, functionalized with microtissues, provides for fast and in depth tissue engineering, without the need for a growth factor and including for scaffolds having a thickness of over 50 μm.

### Example 4

### Osteochondral Implant (In Vivo)

An implant useful for the regeneration of the bone-cartilage unit is prepared as follows.

The nanofibrous three-dimensional scaffold was an electrospun PCL (Poly (ε-caprolactone) nanofibers implant (689±45 μm fiber diameter) with a thickness of 50 μm or 1 mm. The three-dimensional scaffold was covered with an alginate hydrogel comprising chondrocyte microtissues or mesenchymal stem obtained according to the protocol indicated above.

Because the bone lesions were small, it was not necessary to functionalize the scaffold with osteoblasts as these are recruited from the implantation site.

The implant for regeneration of the bone-cartilage unit was then implanted in nude mice for cartilage regeneration as described above.

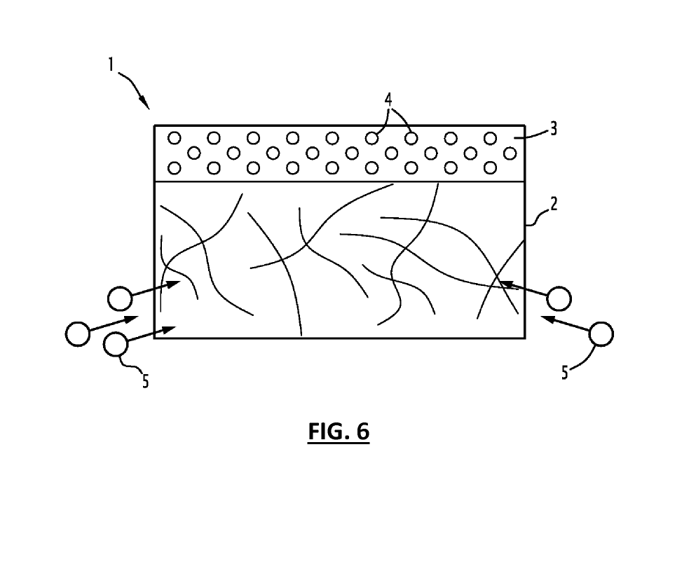

A schematic representation of the resulting implant is shown in FIG. 6. The osteochondral implant (1) comprises a nanofibrous three-dimensional scaffold (2) covered with a hydrogel (alginate) and hyaluronic acid (3) comprising microtissues of chondrocytes or mesenchymal stem cells (4). Osteoblasts (5) do not necessarily need to be seeded as they may be recruited from the implantation site.

1. A biomaterial devoid of a growth factor, comprising:
a three-dimensional scaffold made of a biocompatible polymer; and
living cells,
wherein said living cells are in form of microtissues and the three-dimensional scaffold is a nanofibrous scaffold.

2. The biomaterial according to claim 1, further comprising a hydrogel.

3. The biomaterial according to claim 1, wherein the microtissues comprise osteoblasts, endothelial cells, keratinocytes, myocytes, embryonic stem cells, mesenchymal stem cells and/or chondrocytes.

4. The biomaterial according to claim 1, wherein said biocompatible polymer is selected from poly(ε-caprolactone), collagen, fibrin, poly-(lactic acid), poly(glycolic acid), poly(ethylene glycol) terephthalate, poly(butylene terephthalate) or co-polymers thereof.

5. The biomaterial according to, claim 1, wherein the three dimensional scaffold has a thickness of 50 μm to 2 cm.

6. The biomaterial according to claim 1, wherein the microtissues have a size of 100 to 300 μm.

7. The biomaterial according to claim 1, which is an implant.

8. A method for producing a biomaterial comprising a scaffold made of a nanofibrous biocompatible polymer and living cells, comprising the steps of:
(a) producing a three-dimensional scaffold made of biocompatible polymer; and
(b) contacting said three-dimensional scaffold with microtissues of living cells so as to form a functionalized three-dimensional scaffold.

9. A method according to claim 8, wherein said biocompatible polymer is selected from poly(ε-caprolactone), poly-(lactic acid), poly(glycolic acid), poly(ethylene glycol) terephthalate, poly(butylene terephthalate), collagen, fibrin, hyaluronic acid, chondroitine sulfate, chitosan, copolymers and mixtures thereof.

10. The method according to claim 8, wherein the microtissues are introduced into a hydrogel before step (b).

11. The method of claim 8, wherein step (b) is carried out by injection or deposition of a suspension of microtissues into or onto the three-dimensional scaffold or by dipping the three-dimensional scaffold into said solution or dispersion.

12. A method for the treatment of a bone and/or cartilage defect comprising the use of a biomaterial according to claim 1.

13. The method according to claim 12, for the treatment of a bone and/or cartilage defect in a patient suffering from osteochondritis dissecans, osteonecrosis, osteochondral fracture(s), spinal fusion, a bone and/or cartilage defect due to an injury, a bone and/or cartilage defect due to ageing, a bone and/or cartilage defect necessitating maxillofacial reconstruction, a bone and/or cartilage defect necessitating sinus lift, a bone and/or cartilage defect necessitating alveolar ridge augmentation, or bone and/or cartilage loss due to a tumor.

14. The method according to claim 12, for use in the treatment of a subchondral bone defect or of an osteochondral defect.

15. The method according to claim 12 wherein the biomaterial is used as an implant.
